(19) United States
(12) Patent Application Publication
(10) Pub. No.: US 2006/0036117 A1
(43) Pub. Date: Feb. 16, 2006

(54) Catalyst preparation processes, catalyst regeneration processes, halocarbon production processes, and halocarbon production systems

(76) Inventor: Mitchel Cohn, West Lafayette, IN (US)

Correspondence Address: WELLS ST. JOHN P.S., 601 W. FIRST AVENUE, SUITE 1300, SPOKANE, WA, 99201, US

(21) Appl. No.: 10/916,275

(22) Filed: Aug. 10, 2004

(51) Int. Cl. C07C 17/07

(52) U.S. Cl. 570/101

(57) ABSTRACT

Catalyst preparation and regeneration processes are provided that can include providing a catalyst comprising a first halogen and exposing the catalyst to a reagent comprising a second halogen different from the first halogen. Halocarbon production processes are provided that can include providing a first halocarbon and reacting a halogen exchange reagent and the first halocarbon within a reactor to produce a second halocarbon, the second halocarbon can be a homohalogenated carbon and be essentially free of unsaturated halocarbons. Halocarbon production systems are provided that can include: a halocarbon reagent supply coupled to a reactor; a catalyst supply coupled to the reactor; a halogenation exchange reagent coupled to the reactor; a catalyst regeneration reagent coupled to the reactor; an elemental halogen recovery assembly coupled to the reactor; a catalyst regeneration reagent recovery assembly coupled to the reactor; and a halocarbon recover assembly coupled to the reactor.

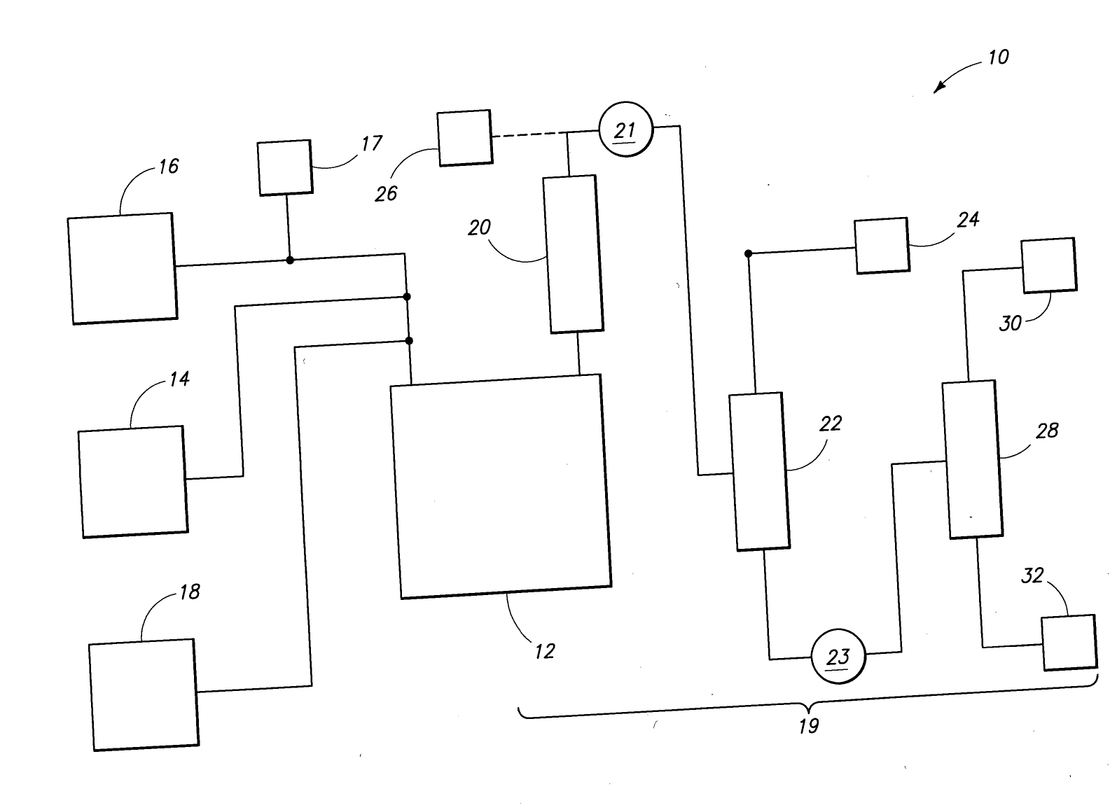

### TECHNICAL FIELD

The invention pertains to processes for preparing catalysts, processes for regenerating catalysts, processes for producing halocarbons, and systems for producing halocarbons.

### BACKGROUND OF THE INVENTION

Halocarbons are utilized as refrigerants, extinguishants, sterilants, and even anesthetics. Hexafluoropropane (CF₃CH₂CF₃, or HFC-236fa) is just one example of a useful halocarbon. The performance of halocarbons can be diminished by impurities and some of these impurities are produced as by-products during the production of the halocarbon. The present disclosure describes processes for preparing catalysts, processes for regenerating catalysts, processes for producing halocarbons, and systems for producing halocarbons that, in exemplary embodiments, can be utilized to produce halocarbons having few, if any, performance diminishing impurities.

While the invention was motivated by addressing the above issues and challenges, it is, of course, in no way so limited. This invention is only limited by the accompanying claims as literally worded and appropriately interpreted in accordance with the doctrine of equivalents.

### SUMMARY OF THE INVENTION

Catalyst preparation processes are provided that include, in an embodiment, providing a catalyst comprising a first halogen. The catalyst is exposed to a reagent comprising a second halogen different from the first halogen to prepare the catalyst. The exposure of the catalyst to the reagent releases the first halogen.

Halocarbon production processes are provided that include, in an embodiment, providing a first halocarbon comprising X. The X of the first halocarbon can represent a first halogen. The first halocarbon is reacted with a halogen exchange reagent within a reactor to produce a second halocarbon comprising Y. Y can be a halogen other than the first halogen. Prior to reacting the first halocarbon and the halogen exchange reagent, the confines of the reactor can be substantially free of released X.

Embodiments also provide processes that include providing a reactor containing a catalyst and preparing the catalyst with a halogenation exchange reagent to form a prepared catalyst. After preparing the catalyst, the prepared catalyst and the halogenation exchange reagent are simultaneously exposed to a reactant halocarbon to produce a saturated halocarbon product essentially free of unsaturated halocarbons.

Embodiments also provide halocarbon production processes that include generating halogens from a liquid phase catalyst to produce a prepared catalyst. The prepared catalyst is exposed to a halogenated carbon to form a homohalogenated carbon.

Catalyst regeneration processes are provided that include, in particular embodiments, providing a mixture comprising a catalyst and a reagent, and exposing the mixture to a halogen until the halogen is essentially no longer consumed by the mixture.

Halocarbon production systems are provided that include, in particular embodiments, a halocarbon reagent supply coupled to a reactor. The reactor can be coupled to a catalyst supply and a halogenation exchange reagent supply. The reactor can also be coupled to a catalyst regeneration reagent supply and an elemental halogen recovery assembly. The reactor can further be coupled to a catalyst regeneration reagent recovery assembly and a halocarbon recovery assembly.

Other aspects and implementations are contemplated.

### BRIEF DESCRIPTION OF THE FIGURE

Preferred embodiments of the invention are described below with reference to the FIGURE of a halocarbon production system according to an embodiment.

### DETAILED DESCRIPTION OF THE PREFERRED EMBODIMENTS

This disclosure of the invention is submitted in furtherance of the constitutional purposes of the U.S. Patent Laws “to promote the progress of science and useful arts” (Article 1, Section 8).

Exemplary processes and systems are described with reference to the FIGURE. Referring to the FIGURE, a halocarbon production system 10 includes a reactor 12 coupled to a reagent halocarbon supply 14 configured to provide halocarbon to reactor 12. Reactor 12 can be configured as a liquid phase reactor. Reactor 12 can be carbon steel and lined with a material such as polytetrafluoroethylene (PTFE) for example. According to exemplary embodiments, reactor 12 can be a carbon steel perfluoroacetate (PFA) lined reactor. Reactor 12 is also coupled to a catalyst supply 16 and a halogenation exchange reagent supply 18 configured to provide catalyst and halogenation exchange reagent to reactor 12. Also coupled to reactor 12 is a catalyst regeneration reagent supply 17 configured to provide catalyst regeneration reagent to reactor 12. The supplies described herein can be configured as individual cylinders or tanks and pressurized with nitrogen and/or pumped to facilitate the charging of their contents to reactor 12. In accordance with exemplary aspects, the cylinders can be situated on a scale to ensure the correct amount of their contents is provided to reactor 12.

System 10 also includes a recovery assembly 19 coupled to reactor 12 and configured to recover released halogens, products, by-products, and reagents from reactor 12. Recovery assembly 19 can include separation assemblies 20, 22, and 28. As depicted in the FIGURE, separation assembly 22 can be coupled to reactor 12 and configured to recover products from reactor 12 for example. Assembly 20 can be configured to recover gaseous products by refluxing a liquid phase back to the reaction contained in reactor 12 whereby the majority of the reactants remain in reactor 12 and the products are passed to separation assembly 20. Halogen recovery assembly 24 and catalyst regeneration reagent recovery assembly 26 can be coupled to separation assembly 22. Separation assembly 22 can also be coupled to separation assembly 28 which can be coupled to halocarbon product recovery assembly 32 and halogenation exchange reagent recovery assembly 30.

Embodiments of system 10 can be utilized to facilitate catalyst preparation processes. According to an exemplary embodiment, catalyst supply 16 contains a catalyst that can be provided to reactor 12 and exposed to halogenation reagent provided from halogenation reagent supply 18. The catalyst can be of the formula M^{a}Zₐ with M representing a metal, Z representing one or more halogens and “a” representing the oxidation state of the metal. The catalyst can be suitable for use with liquid phase halogen exchange processes. M can include antimony (Sb) in exemplary embodiments. Z can be one or more halogens, including exemplary halogens F and Cl. In other embodiments, the catalyst can include SbY_{b}Z_{(5-b)}, with Y being a first halogen and Z being another halogen other than Y and “b” being an integer less than 5. The catalyst can include SbF_{b}Cl_{(5-b)} in other aspects. Typical oxidation states of Sb are 3 and 5 thereby leaving the accumulation of halogens associated with the Sb catalyst at 3 and 5. The catalyst can be SbCl₅ prior to preparation, but prepared catalyst will typically have at least one other halogen other than the Cl present.

The catalyst can be provided from catalyst supply 16 to reactor 12. Once a predetermined amount of catalyst is provided to reactor 12, in exemplary embodiments, halogenation exchange reagent from halogenation exchange reagent supply 18 can be added to reactor 12. The halogenation exchange reagent can include a halogen different from the halogens present in the catalyst. The catalyst can include only chlorine halogens and the halogen exchange reagent can include only fluorine halogens for example. In exemplary embodiments the halogen exchange reagent is HF and the catalyst is SbCl₅.

Upon addition of the HF exchange reagent to reactor 12 containing the SbCl₅ catalyst, reactor 12 can be heated to a temperature of 80-90° C. When heated to this temperature, halogen in the form of HCl can be released from the catalyst and recovered from reactor 12 via recovery assemblies 19. In accordance with the exemplarily depicted embodiments of the FIGURE, separation assembly 20 can be coupled to the upper portion of reactor 12 and separation assembly 22 can be coupled to the upper portion of separation assembly 20 to facilitate the recovery of released halogen such as HCl by halogen recovery assembly 24 coupled to an upper portion of separation assembly 22.

According to exemplary aspects, the halogen exchange reagent can be continually added to reactor 12 containing the catalyst, and heated until the released halogen is essentially no longer recovered by recovery assembly 24. For example and by way of example only, an assay of the contents of recovery assembly 24 yielding less than percentage quantities of the released halogen can be an indication that the released halogen is no longer recovered. In exemplary embodiments, to prepare reactor 12 or the catalyst within reactor 12, assembly 24 is monitored until essentially none of the released halogen is recovered. At this point the contents of reactor 12 can be considered essentially free of the released halogen.

In exemplary embodiments, the released halogen can include the halogens of the catalyst prior to preparation. For example and by way of example only, the released halogen can include Cl in the form of HCl that is evolved from reactor 12 when SbCl₅ is heated in the presence of HF. The prepared catalyst can include SbF₃Cl₂. In accordance with exemplary aspects, a greater excess of the halogenation exchange reagent, as compared to the catalyst, is contained within reactor 12 throughout the preparation of the catalyst and throughout at least some of the halocarbon production processes described below.

Upon preparation of the catalyst, a reagent halocarbon from reagent halocarbon supply 14 can be charged to reactor 12. In accordance with the exemplarily depicted embodiments of the FIGURE, the conduit from reagent halocarbon supply 14 to reactor 12 can be protected from reverse flow by a disk back check valve configured between supply 14 and reactor 12. The reagent halocarbon can comprise X, with X representing a first halogen. The reagent halocarbon can include CCl₃CH₂CCl₃ in exemplary embodiments. CCl₃CH₂CCl₃ can be acquired from a homoligation such as the homoligation of carbon tetrachloride and vinylidene chloride. CCl₃CH₂CCl₃ can be charged to reactor 12 containing: the halogenation exchange reagent HF; the prepared catalyst; and essentially no elemental halogen from the catalyst. In exemplary embodiments, the reagent halocarbon can be simultaneously exposed to the catalyst and the halogenation exchange reagent within reactor 12.

Upon exposure to the prepared catalyst and the halogenation exchange reagent to the reagent halocarbon, a product halocarbon can be produced. The product halocarbon can be collected from reactor 12, for example, by utilizing recovery assembly 19. The product halocarbon, in exemplary aspects, can contain a halogen of the halogen exchange reagent. The product halocarbon can be saturated and be essentially free of unsaturated halocarbons. In particular aspects, the reagent halocarbon can be CCl₃CH₂CCl₃ and the product halocarbon can be essentially free of unsaturated halocarbons such as CF₃CHCF₂. According to exemplary embodiments, the saturated halocarbon product can be a homohalogenated carbon such as CF₃CH₂CF₃. The product halocarbon can contain less than 100 parts per million unsaturated halocarbons and, in other aspects, the amount of unsaturated halocarbons cannot be detected. During reaction of reagent halocarbon to form the product halocarbon, reactor 12 can be maintained between 6.5×10⁵ Pa and 7.2×10⁵ Pa.

Upon production of product halocarbon from reactor 12, product halocarbon can be isolated utilizing recovery assembly 19. Recovery assembly 19 can include a separation assembly 20 coupled to the upper portion of reactor 12. In exemplary embodiments, separation assembly 20 can be a condenser and/or distillation apparatus. For example and by way of example only, assembly 20 can be a 6 cm outside diameter lined schedule 40 pipe packed with 1 cm PFA tubing cut into approximately 1.3 cm pieces and supported by a fabricated 0.3 cm PTFE sheet with a crosscut support design having a PTFE mesh loosely wadded at the upper portion. Temperature of the distillation apparatus can be between 15-90° C. According to exemplary embodiments, reactor 12 can have a temperature of 90° C. and assembly 20 can have an inlet of 70° C., a midpoint of 67° C., and an outlet of 15° C.

A product mixture 21 recovered at the upper portion of assembly 20 can include product halocarbon, halogenation exchange reagent, and any other halogenation exchange reagent by-products such as HCl, which is the case when the halogenation exchange reagent includes HF and the reagent halocarbon includes Cl. Mixture 21 can be transferred to separation assembly 22 where the by-product can be recovered by halogen recovery assembly 24 at the upper portion of assembly 22, and a mixture 23 of excess halogenation exchange reagent and product halocarbon can be recovered from a lower portion of separation assembly 22. Separation assembly 22 can be a distillation apparatus.

Mixture 23 can be further purified to isolate the product halocarbon. According to the exemplarily depicted embodiment of the FIGURE, mixture 23 can be transferred to separation assembly 28. Separation assembly 28 can be a phase separator or a distillation apparatus. The halogenation exchange reagent can be recovered by halogenation exchange reagent recovery assembly 30 coupled to an upper portion of assembly 28, and product halocarbon can be recovered by product halocarbon recovery assembly 32. Recovery assembly 30 can be configured to recycle recovered halogenation exchange reagent to reactor 12 and assembly 32 can be configured to further purify the product halocarbon.

According to another embodiment, the catalyst within reactor 12 can be regenerated by exposing the catalyst to a catalyst regenerating reagent from catalyst regenerating reagent supply 17. The catalyst regenerating reagent can include diatomic reagents or reagents having similar elements to the catalyst within catalyst supply 16 for example. According to exemplary aspects, where the catalyst within catalyst supply 16 is SbCl₅ and the prepared catalyst within reactor 12 is SbCl₂F₃, the catalyst regenerating reagent can include Cl₂. The catalyst regenerating reagent and the reactant halocarbon can comprise one like halogen in exemplary embodiments. For example, where the reactant halocarbon is CCl₃CH₂CCl₃, the regenerating reagent can be Cl₂.

The catalyst within reactor 12 can be regenerated by exposing the contents of reactor 12 to the catalyst regenerating reagent until the reagent is no longer consumed by the mixture within reactor 12. In exemplary embodiments reactor 12 can contain the catalyst and the halogenation exchange reagent. The catalyst can be considered regenerated, upon exposure of these contents to the regenerating reagent, when the regenerating reagent evolves from reactor 12 and can be recovered by assembly 19. For example, in the case where regenerating reagent includes Cl₂, separation assembly 20 can facilitate the recovery of regenerating reagent directly from reactor 12. The recovered regenerating reagent can include the catalyst regenerating reagent and/or elements of the regenerating reagent such as Cl and/or diatomic Cl₂ for example. Upon regeneration, the catalyst contained within reactor 12 can be considered prepared catalyst and utilized to produce halocarbons according to the methods described herein and other methods.

In compliance with the statute, the invention has been described in language more or less specific as to structural and methodical features. It is to be understood, however, that the invention is not limited to the specific features shown and described, since the means herein disclosed comprise preferred forms of putting the invention into effect. The invention is, therefore, claimed in any of its forms or modifications within the proper scope of the appended claims appropriately interpreted in accordance with the doctrine of equivalents.

1. A catalyst preparation process comprising:
providing a catalyst comprising a first halogen; and
exposing the catalyst to a reagent comprising a second halogen different from the first halogen to prepare the catalyst, wherein the exposing the catalyst to the reagent releases the first halogen.

2. The process of claim 1 further comprising recovering the first halogen after the exposing the catalyst to the reagent.

3. The process of claim 1 wherein the exposing the catalyst to the reagent comprises continually adding the reagent to a reactor containing the catalyst and further comprises recovering the first halogen from the reactor.

4. The process of claim 3 wherein the catalyst comprises SbCl₅, the reagent comprises HF, and the recovering the first halogen from the reactor comprises recovering HCl.

5. The process of claim 3 further comprising stopping the continually adding the reagent to the reactor when the first halogen can essentially no longer be recovered from the reactor.

6. The process of claim 1 wherein the first halogen is Cl and the second halogen is F.

7. The process of claim 1 wherein the catalyst comprises Sb and at least Cl.

8. The process of claim 1 wherein the catalyst comprises SbY_{b}Z_{(5-b)}, wherein Y is the first halogen, Z is another halogen other than Y, and b≦5.

9. A halocarbon production process comprising:
providing a first halocarbon comprising X, wherein X represents a first halogen; and
reacting a halogen exchange reagent and the first halocarbon within a reactor to produce a second halocarbon comprising Y, Y being another halogen than the first halogen, wherein the confines of the reactor are substantially free of released X prior to the reacting.

10. The process of claim 9 wherein the first halogen comprises Cl.

11. The process of claim 10 wherein the first halocarbon comprises CCl₃CH₂CCl₃.

12. The process of claim 11 wherein the halogen exchange reagent comprises HF and the second halocarbon comprises CF₃CH₂CF₃.

13. The process of claim 9 wherein the halogen exchange reagent comprises H.

14. The process of claim 9 wherein the halogen exchange reagent comprises HY.

15. The process of claim 14 wherein the first halocarbon comprises CCl₃CH₂CCl₃ and the second halocarbon comprises CF₃CH₂CF₃.

16. The process of claim 9 wherein the reacting is performed in the presence of a catalyst.

17. The process of claim 16 wherein the catalyst comprises M^{a}Zₐ, wherein M represents a metal, Z represents one or more halogens, and “a” represents an oxidation state of the metal.

18. The process of claim 17 wherein M comprises Sb.

19. The process of claim 16 wherein the catalyst comprises SbF_{b}Cl_{(5-b)}, wherein b≦5.

20. A halocarbon production process comprising:
providing a reactor containing a catalyst;
preparing the catalyst with a halogenation exchange reagent to form a prepared catalyst; and
after preparing the catalyst, simultaneously exposing the prepared catalyst and the halogenation exchange reagent to a reactant halocarbon to produce a saturated halocarbon product essentially free of unsaturated halocarbons.

21. The process of claim 20 wherein the catalyst comprises a liquid phase halogenation exchange catalyst.

22. The process of claim 20 wherein the halogenation exchange reagent comprises H.

23. The process of claim 20 wherein the prepared catalyst comprises Sb, the halogenation exchange reagent comprises HF, the reactant halocarbon comprises Cl, and the saturated halocarbon product comprises F.

24. The process of claim 23 wherein the prepared catalyst comprises Sb and F, the reactant halocarbon comprises CCl₃CH₂CCl₃, and the saturated halocarbon product is essentially free of CF₃CHCF₂.

25. The process of claim 20 further comprising, after simultaneously exposing the catalyst and the halogenation exchange reagent to the reactant halocarbon, regenerating the prepared catalyst, the regenerating comprising exposing the prepared catalyst to a catalyst regenerating reagent, wherein both the catalyst regenerating reagent and the reactant halocarbon comprise at least one like halogen.

26. The process of claim 25 wherein the catalyst regenerating reagent comprises Cl₂.

27. A halocarbon production process comprising:
generating halogens from a liquid phase catalyst to produce a prepared catalyst; and
exposing a halogenated carbon to the prepared catalyst to form a homohalogenated carbon.

28. The process of claim 27 wherein the liquid phase catalyst comprises SbCl₅ and the generating halogens comprises generating HCl.

29. The process of claim 27 wherein the prepared catalyst comprises Sb and F.

30. The process of claim 27 wherein the halogenated carbon comprises Cl and the homohalogenated carbon comprises F.

31. The process of claim 27 wherein the liquid phase catalyst comprises SbCl₅, the halogenated carbon comprises CCl₃CH₂CCl₃ and the homohalogenated carbon comprises CF₃CH₂CF₃.

32. The process of claim 31 wherein the generating halogens from a liquid phase catalyst to produce a prepared catalyst, comprises adding HF to a reactor containing the SbCl₅ to produce the prepared catalyst, wherein the prepared catalyst comprises Sb and F.

33. The process of claim 32 wherein the exposing the halogenated carbon to the prepared catalyst comprises simultaneously exposing both the prepared catalyst and the HF to the halogenated carbon.

34. A catalyst regeneration process comprising:
providing a mixture comprising a liquid phase catalyst and a halogenation exchange reagent; and
exposing the mixture to a halogen until the halogen is essentially no longer consumed by the mixture.

35. The process of claim 34 wherein the catalyst comprises M^{a}X_{(a-b)}Y_{b}, wherein M represents a metal, X represents a first halogen, Y represents a second halogen different from the first halogen, “a” represents the oxidation state of the metal, and “b” is an integer less than or equal to “a”.

36. The process of claim 35 wherein the M is Sb, the X is Cl, and the Y is F.

37. The process of claim 34 wherein the catalyst comprises SbF_{b}Cl_{(5-b)} and b≦5.

38. The process of claim 37 wherein the reagent comprises HF and the halogen comprises Cl₂.

39. The process of claim 34 wherein the catalyst comprises Sb, the reagent comprises H, and the halogen comprises a diatomic halogen.

40. The process of claim 34 wherein the exposing the mixture to a halogen until the halogen is essentially no longer consumed by the mixture comprises continually adding the halogen to a reactor containing the mixture until the halogen evolves from the mixture.

41. The process of claim 40 wherein the halogen evolving from the mixture and the halogen exposed to the mixture are in the same form.

42. The process of claim 41 wherein the form is diatomic.

43. A halocarbon production system comprising:
a halocarbon reagent supply coupled to a reactor;
a catalyst supply coupled to the reactor;
a halogenation exchange reagent coupled to the reactor;
a catalyst regeneration reagent coupled to the reactor;
an elemental halogen recovery assembly coupled to the reactor;
a catalyst regeneration reagent recovery assembly coupled to the reactor; and
a halocarbon recovery assembly coupled to the reactor.

44. The system of claim 43 wherein the halocarbon reagent comprises CCl₃CH₂CCl₃.

45. The system of claim 43 wherein the catalyst comprises SbCl₅.

46. The system of claim 43 wherein the halogenation exchange reagent comprises HF.

47. The system of claim 43 wherein the catalyst regeneration reagent comprises Cl₂.

48. The system of claim 43 wherein the elemental halogen comprises HCl and the elemental halogen recovery assembly comprises a condenser.

49. The system of claim 43 wherein the catalyst regeneration reagent recovery assembly comprises a distillation apparatus.

50. The system of claim 43 wherein the halocarbon comprises CF₃CH₂CF₃ and the halocarbon recovery assembly comprises a distillation apparatus.

51. The system of claim 43 wherein the recovery assemblies share portions of the same assembly.
